(19) United States
(12) Patent Application Publication
(10) Pub. No.: US 2013/0317319 A1
(43) Pub. Date: Nov. 28, 2013

(54) PHYSIOLOGY MONITORING SYSTEM AND PHYSIOLOGY MONITORING METHOD

(71) Applicants: ACCTON TECHNOLOGY CORPORATION, Hsinchu (TW); NATIONAL CHENG KUNG UNIVERSITY, Tainan City (TW)

(72) Inventors: Yueh-Min HUANG, Tainan City (TW); Chin-Feng LAI, Kaohsiung City (TW); Ying-Xun LAI, Kaohsiung City (TW); Sung-Yen CHANG, Yilan County (TW); Fuh-Jang LIN, Hsinchu City (TW)

(73) Assignees: NATIONAL CHENG KUNG UNIVERSITY, Tainan City (TW); ACCTON TECHNOLOGY CORPORATION, Hsinchu (TW)

(21) Appl. No.: 13/872,151

(22) Filed: Apr. 28, 2013

(30) Foreign Application Priority Data
May 23, 2012 (TW) 101118352

(51) Int. Cl. A61B 5/00; A61B 5/024; A61B 5/021; A61B 5/01; A61B 5/145

(52) U.S. Cl. CPC A61B 5/7271; A61B 5/01; A61B 5/14542; A61B 5/021; A61B 5/024
USPC 600/301; 600/300; 600/549; 600/364; 600/485; 600/508

(57) ABSTRACT

A physiology monitoring system and a physiology monitoring method are disclosed. The physiology monitoring system includes a physiological information sensor, a data analysis device and an application service system. The physiological information sensor is suitable for sensing physiological information of a user. The data analysis device is suitable for receiving data of the physiological information from the physiological information sensor, calculating a plurality of data features of the data, determining whether each data feature has an alert condition event, calculating occurrence probabilities of a plurality of critical condition events or a plurality of physiology condition events by using the corresponding alert condition event or events, and determining a critical condition or a physiology condition of the user according to the occurrence probabilities. The application service system can provide the user with a service according to the critical condition or a physiology condition of the user.

### RELATED APPLICATIONS

This application claims priority to Taiwan Application Serial Number 101118352, filed May 23, 2012, which is herein incorporated by reference.

### BACKGROUND

1. Field of Invention

The invention relates to a monitoring system. More particularly, the invention relates to a physiology monitoring system and a physiology monitoring method.

2. Description of Related Art

With the development of information technology and the increasing popularity of Internet, the smart lifestyles, such as home digitalization, medial care and smart learning, have been gradually valued and adopted by the public. Accordingly, health care equipments are not exclusive to hospitals, and a lot of household medical equipments are developed and produced in succession. Additionally, diverse learning methods arise because of theintroduction of digital learning.

In order to match with the smart lifestyles, many devices and facilities have been developed currently. Most of these devices and facilities measure information of users through physiological sensors and provide the corresponding application services.

However, all these physiological sensors are installed in peripheral devices of a computer. For the same physiological sensing demand, these peripheral devices cannot share the same physiological sensor. However, too many physiological sensors unavoidably have reduplicated functions. Accordingly, it is a burden for the users to buy the peripheral devices, thereby reducing purchase intentions of the users.

Moreover, these physiological sensors are merely used to measure the physiological information of the user, so that the physiological sensors cannot obtain current physiology condition of the user to immediately provide corresponding alert information. Therefore, a physiology monitoring system and a physiology monitoring method are needed to integrate the physiological information sensor with the smart living to provide the users with more humanized and friendly information and services.

### SUMMARY

Accordingly, an aspect of the invention is to provide a physiology monitoring system and a physiology monitoring method, in which a physiological information sensor may be attached to an outer side surface of a device being used by a user through a connector. Therefore, the physiological information sensor can be applied to various devices according to the demand of the user, thereby greatly increasing the application of the physiological information sensor and lowering the burden of the user.

A further aspect of the invention is to provide a physiology monitoring system and a physiology monitoring method, which can integrate a physiological information sensor and a data analysis device for physiological information with an application service system effectively. Accordingly, the sensed physiological information can be immediately analyzed and then the physiology condition of a user can be determined, so as to provide an application service needed by the user to achieve the effect of integrating the physiological information sensor with life.

According to the aforementioned aspects of the present invention, a to physiology monitoring system is provided. The physiology monitoring system includes a physiological information sensor, a data analysis device and an application service system. The physiological information sensor is suitable for sensing at least one physiological information of a user. The data analysis device is suitable for receiving a plurality of data of the physiological information from the physiological information sensor, calculating a plurality of data features of the data of the at least one physiological information, determining whether each data feature has an alert condition event, calculating occurrence probabilities of a plurality of critical condition events or a plurality of physiology condition events by using the corresponding alert condition event or events, and determining a critical condition or a physiology condition of the user according to the occurrence probabilities of the critical condition events or the physiology condition events. The application service system is suitable for providing the user with a service according to the critical condition or a physiology condition of the user.

According to one embodiment of the present invention, the aforementioned physiological information sensor includes a casing, a physiological information sensing module, a message transmission module, a system management module and a connector. The physiological information sensing module is disposed within the casing and is suitable for sensing at least one physiological information. The message transmission module is disposed within the casing and is suitable for transmitting the received data to the data analysis device. The system management module is disposed within the casing and is suitable for collecting the data of the at least one physiological to information and transmitting the data to the message transmission module. The connector is disposed on an outer side surface of the casing and is suitable for connecting the casing to a device being used by the user.

According to another embodiment of the invention, the aforementioned application service system is a health care system and is suitable for providing the user with a health care service according to the critical condition of the user. Additionally, the aforementioned at least one physiological information includes body temperature, blood oxygen concentration, blood pressure and/or heartbeat, and the data features include a maximum value, a minimum value, an average value, a root mean square value, a standard deviation, information entropy and/or a frequency.

According to still another embodiment of the present invention, the aforementioned application service system is a learning analysis system and is suitable for facilitating learning efficiency of the user according to the physiology condition of the user. Additionally, the aforementioned at least one physiological information includes body temperature, blood oxygen concentration and/or pressure, and the data features include an average value, a duration time, an amount and/or a discrete value.

According to the aforementioned aspects of the present invention, a physiology monitoring method is further provided, which includes the following steps. A physiological information sensor is used to sense at least one physiological information of a user. This physiological information sensor is used to compare a plurality of data of the at least one physiological information being sensed. A data analysis device is used to calculate a plurality of data features corresponding to the data of the at least one physiological information through the data of the at least one physiological information. The data analysis device is used to determine whether each data feature has an alert condition event. When one alert condition event or a plurality of alert condition events occur among the data features, the data analysis device is used to calculate occurrence probabilities of corresponding critical condition events or corresponding physiology condition events according to the alert condition event or events. The data analysis device is used to determine a critical condition or a physiology condition of the user according to the occurrence probabilities of the critical condition events or physiology condition events corresponding to the alert condition event or events. The data analysis device is used to transmit the critical condition or physiology to an application service system.

According to one embodiment of the present invention, the aforementioned physiological information sensor includes a casing, and a physiological information sensing module, a message transmission module and a system management module disposed within the casing.

According to another embodiment of the present invention, the aforementioned system management module includes a timing unit, a signal conversion unit and a comparison unit. Moreover, the step of comparing the data includes the following steps. The timing unit is used to periodically require the physiological information sensing module to sense the at least one physiological information of the user. The signal conversion unit is used to convert the data from an analogue model to a digital model. The comparison unit is used to compare the converted data to determine whether each datum is changed with respect to a previous datum of the at least one physiological information.

According to still another embodiment of the present invention, when each data feature does not have any alert condition event, the data analysis device is used to display the data; or the data analysis device is used to transmit the data to the application service system and the data are displayed by the application service system.

According to yet still another embodiment of the present invention, when each datum complies with a historical record, the aforementioned physiology monitoring method returns to the step of sensing the at least one physiological information of the user. Additionally, when each datum does not comply with the historical record, the historical record is updated with each datum by the data analysis device.

According to further another embodiment of the present invention, the aforementioned application service system is a health care system. The physiological information sensor is used to sense body temperature, blood oxygen concentration, blood pressure and/or heartbeat of the user. The data analysis device is used to calculate a maximum value, a minimum value, an average value, a root mean square value, a standard deviation, information entropy and/or a frequency corresponding to the data of the aforementioned at least one physiological information.

According to further another embodiment of the present invention, the aforementioned application service system is a learning analysis system. The physiological information sensor is used to sense body temperature, blood oxygen concentration and/or pressure of the user. The data analysis device is used to calculate an average value, a duration time, an amount and/or a discrete value corresponding to the data of the aforementioned at least one physiological information.

According to further another embodiment of the present invention, the aforementioned step of using the data analysis device to determine whether each data feature has an alert condition event is performed by determining whether each data feature exceeds a corresponding predetermined alert value. When one of the data features exceeds the corresponding predetermined alert value, the one of the data features has the alert condition event.

According to further another embodiment of the present invention, the step of using the data analysis device to calculate the occurrence probabilities of the corresponding critical condition events or the physiology condition events according to the alert condition event or events includes the following steps. The occurrence probability of each critical condition event or physiology condition event under the alert condition event or events is found according to a historical record. The possible occurrence probability of each critical condition event or physiology condition event corresponding to the alert condition event or events and the occurrence probabilities of the alert condition event or events under each critical condition event or physiology condition event are counted. The occurrence probabilities of the critical condition events or the physiology condition events are multiplied by the occurrence probabilities of the corresponding alert condition event or events, and then is divided by the possible occurrence probability of each critical condition event or physiology condition event corresponding to the alert condition event or events.

According to further another embodiment of the present invention, when the aforementioned alert condition event lasts more than a predetermined duration time, the data analysis device determines that the critical condition event or the physiology condition event occurs.

### BRIEF DESCRIPTION OF THE DRAWINGS

In order to make the foregoing as well as other purposes, features, advantages and embodiments of the present invention more readily appreciated, the accompanying drawings are described as follows:

FIG. 1 illustrates a block diagram of a physiology monitoring system in accordance with one embodiment of the present invention;

FIG. 2 illustrates a block diagram of a physiological information sensor in accordance with one embodiment of the present invention;

FIG. 3 illustrates a schematic diagram of a physiological information sensor arranged on a mouse in accordance with one embodiment of the present invention; and

FIG. 4 illustrates a flowchart showing a physiology monitoring method in accordance with one embodiment of the present invention.

### DETAILED DESCRIPTION

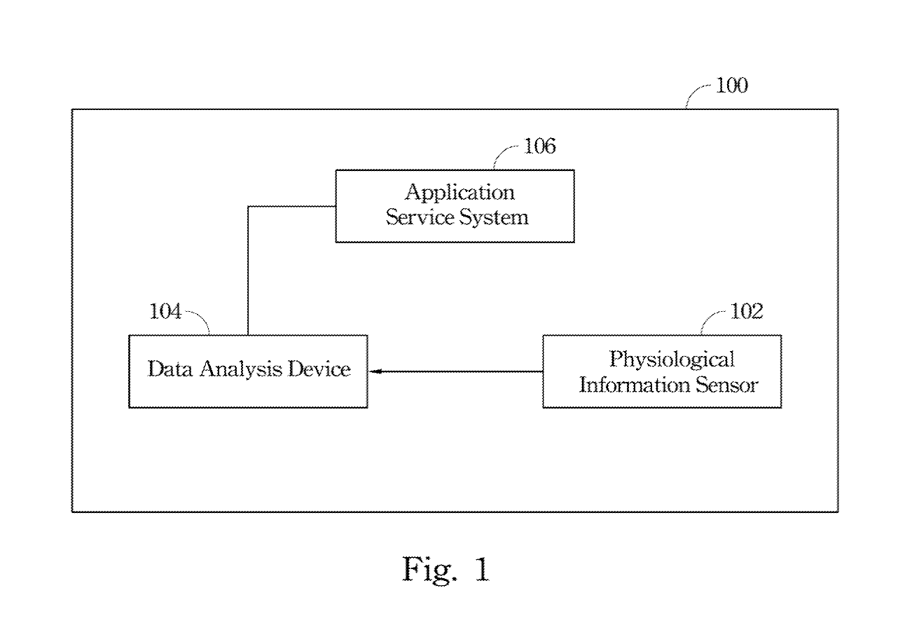

Refer to FIG. 1. FIG. 1 illustrates a block diagram of a physiology monitoring system in accordance with one embodiment of the present invention. In the present embodiment, a physiology monitoring system 100 mainly includes a physiological information sensor 102, a data analysis device 104 and an application service system 106. The physiological information sensor 102 is suitable for sensing one or more physiological information of a user. The data analysis device 104 is suitable for analyzing data of the physiological information sensed by the physiological information sensor 102 and determining the physiology condition of the user according to the analysis result. The application service system 106 is suitable for providing a service needed by the user according to the physiology condition of the user.

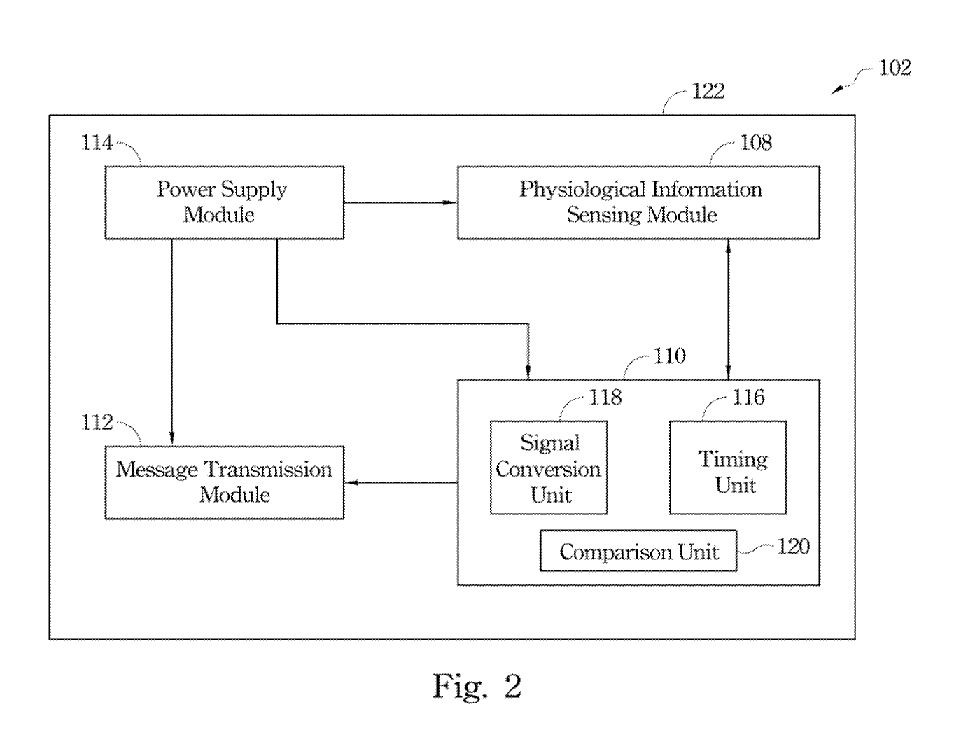

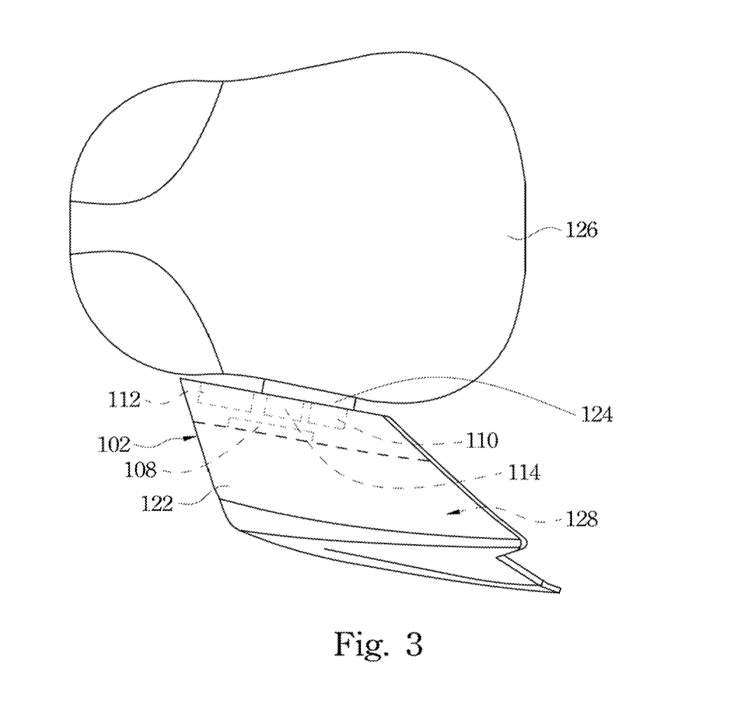

Refer to FIG. 2 and FIG. 3 simultaneously. FIG. 2 and FIG. 3 respectively illustrate a block diagram of a physiological information sensor and a schematic diagram of the physiological information sensor arranged on a mouse in accordance with one embodiment of the present invention. In one exemplary embodiment, as shown in FIG. 2, the physiological information sensor 102 mainly includes a casing 122, a physiological information sensing module 108, a system management module 110 and a message transmission module 112. As shown in FIG. 3, the physiological information sensing module 108, the system management module 110 and the message transmission module 112 are all disposed within the casing 122. With the enclosure of the casing 122, the physiological information sensing module 108, the system management module 110 and the message transmission module 112 are protected from damage. In one exemplary embodiment, the casing 122 may be a long hollow casing with an accommodation hole 128 to accommodate a finger of the user, e.g., a thumb or a middle finger, for measuring the physiological information of the user.

In the embodiment, according to the usage demand, the physiological information sensor 102 may further include a connector 124 selectively. As shown in FIG. 3, the connector 124 may be disposed on an outer side surface of the casing 122. With the connector 124, the casing 122 can be connected to a device being used by the user, e.g., a mouse 126 shown in FIG. 3, to provide the physiological information measurement service. In one exemplary embodiment, for example, the connector 124 may be a double sticky tape, which can be pasted repeatedly. Accordingly, with the connection of the double sticky tape, the physiological information sensor 102 can be arranged on other devices according to different usage demands of the user.

The physiological information sensing module 108 is suitable for sensing at least one physiological information of the user, e.g., body temperature, blood oxygen concentration, blood pressure, heartbeat, pressure, etc. In one exemplary embodiment, the physiological information sensing module 108 is disposed within the casing 122 adjacent to the accommodation hole 128 of the casing 122, so that the physiological information of the user can be sensed through the finger of the user placed in the accommodation hole 128.

The system management module 110 may be suitable for collecting data of the physiological information sensed by the physiological information sensing module 108, and transmitting the collected data of the physiological information to the message transmission module 112. As shown in FIG. 2, in one exemplary embodiment, the system management module 110 may include a timing unit 116 and a comparison unit 120. With the timing unit 116, the system management module 110 can periodically require the physiological information sensing module 108 to sense the physiological information and retrieve the data of the physiological information sensed by the physiological information sensing module 108. The comparison unit 120 can be used to compare to determine whether a datum of the physiological information is changed with respect to a previous datum. When this datum of the data of the physiological information is changed with respect to the previous datum, the system management module 110 will transmit the datum to the message transmission module 112. On the other hand, when the datum of the data of the physiological information is not changed with respect to the previous datum, the system management module 110 will not transmit the datum to the message transmission module 112, thereby preventing electric power from being consumed due to too many data transmissions.

In one exemplary embodiment, the system management module 110 may further include a signal conversion unit 118 selectively according to formats of the collected data of the physiological information, e.g., an analogue model. The signal conversion unit 118 is suitable for converting the data of the physiological information collected by the system management module 110 from an analogue model to a digital model, for facilitating the message transmission module 112 to transmit the data signal. In the exemplary embodiment, the data of the physiological information collected by the system management module 110 are firstly converted from an analogue model to a digital model by the signal conversion unit 118, and then the converted data are compared by the comparison unit 120.

The message transmission module 112 can receive the data of the physiological information transmitted by the system management module 110 and then transmit the received data of the physiological information to the data analysis device 104 through a wire or wireless transmission way, for example. In one exemplary embodiment, the message transmission module 112 can transmit the received data of the physiological information to the data analysis device 104 through a wireless transmitter, e.g., a Zigbee wireless transmitter.

In one exemplary embodiment, the physiological information sensor 102 may further include a power supply module 114 selectively according to the electric power demand. The power supply module 114 may be electrically connected with the physiological information sensing module 108, the message transmission module 112 and the system management module 110, to supply the physiological information sensing module 108, the message transmission module 112 and the system management module 110 with electric power. In some examples, the power supply module 114 may consist of typical batteries.

The data analysis device 104 can analyze the received data of the physiological information and determine the physiology condition of the user according to an analysis result of the data, e.g., determine that what kind of critical condition event or physiology condition event of the user may occur. In one exemplary embodiment, the data analysis device 104 may be a computer, a mobile device, a processor, a microcontroller, an operation-executable and/or controllable chip set that can execute operation and/or control, or an instrument that can manage and calculate the data. Refer to FIG. 1 again. The data analysis device 104 can further transmit the analysis result to the application service system 106. The application service system 106 can receive the data of the physiological information analyzed by the data analysis device 104 and the physiology condition of the user, and display the related physiological information of the user or provide the user with the corresponding service according to the physiology condition of the user. In another exemplary embodiment, the data analysis device 104 also can display the related physiological information of the user.

In an exemplary example, the application service system 106 may be a health care system, and may provide the user with a health care service according to the physiology condition of the user provided by the data analysis device 104. In the exemplary example, the physiological information sensor 102 can measure the physiological information of the user, such as body temperature, blood oxygen concentration, heartbeat, pressure, etc. However, the exemplary example may not be limited to the aforementioned specific physiological information, and different kinds of physiological information may be measured by modifying the physiological information sensing module 108.

Additionally, in the exemplary example, the data analysis device 104 can analyze various kinds of received physiological information of the user and calculate data features, such as a maximum value, a minimum value, an average value, a root mean square value, a standard deviation, information entropy, a frequency, etc., corresponding to various physiological information groups according to the physiological information groups. When any data feature of the physiological information group exceeds a predetermined alert value, the data analysis device 104 will determine that an alert condition event occurs. Then, the data analysis device 104 determines the critical condition event according to the alert condition event. If the data analysis device 104 determines that the user is in the critical condition now, then the data analysis device 104 transmit an evaluation result to the application service system 106 to inform that the critical condition event may be happened to the user. In another exemplary example, the application service system 106 may be a learning analysis system and can assist the teaching according to the physiology condition of the user provided by the data analysis device 104, so as to facilitate the learning efficiency of the user. In the exemplary example, the physiological information sensor 102 can measure the physiological information of the user, such as body temperature, pressure applied to the mouse, blood oxygen concentration, etc. Similarly, the exemplary example may not be limited to the aforementioned specific physiological information, and different kinds of physiological information may be measured by modifying the physiological information sensing module 108.

In the exemplary example, the data analysis device 104 can analyze various received physiological information groups of the user, such as body temperature, pressure applied to the mouse, blood oxygen concentration, etc., and calculate the data features, such as a duration time, an amount, a discrete value, an average value, etc., corresponding to the various physiological information groups. When any data feature of the physiological information group exceeds a predetermined alert value, the data analysis device 104 will determine that an alert condition event occurs. Then, the data analysis device 104 determines the physiology condition event according to the alert condition event. According to the determined physiology condition event, the data analysis device 104 transmits an evaluation result to the application service system 106 to inform that the physiology condition event may be happened to the user, so as to provide a corresponding action responsive to the physiology condition event. For example, if the physiology condition event is determined as tiredness, the user may be required to stand up to do some activities.

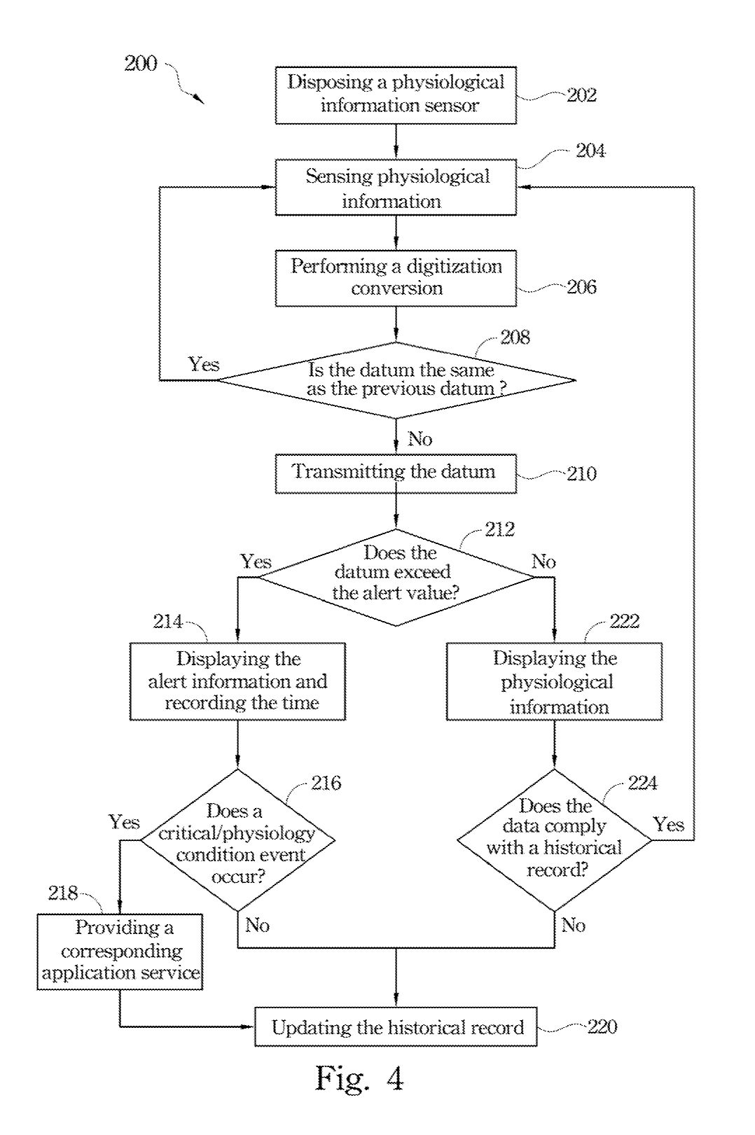

Refer to FIG. 1 through FIG. 4 simultaneously. FIG. 4 illustrates a flowchart showing a physiology monitoring method in accordance with one embodiment of the present invention. In the embodiment, when a physiology monitoring method 200 is performed, as described in a step 202, the physiological information sensor 102 may be disposed on a device being used by a user, e.g., the mouse 126 shown in FIG. 3, by, for example, the connector 124. In the step 202, when the user desires to use a certain device, the user may dispose the physiological information sensor 102 on the device through the connector 124, so as to start the function of the physiological information sensor 102 to sense the physiological information of the user.

Then, as described in a step 204, the physiological information sensor 102 is used to sense one or more kinds of physiological information of the user, such as body temperature, blood oxygen concentration, blood pressure, heartbeat, pressure, etc. In the step 204, when the user uses the device, the user can place his thumb or middle finger in the accommodation hole 128 of the casing 122 of the physiological information sensor 102 on an outer side surface of the device, as shown in FIG. 3. Next, the system management module 110 of the physiological information sensor 102 can use the timing unit 116 of the system management module 110 to periodically transmit a command to require the physiological information sensing module 108 to sense the physiological information of the user and retrieve the data of the physiological information sensed by the physiological information sensing module 108.

Then, when the data of the physiological information collected by the system management module 110 are in an analogue format, the system management module 110 may further include the signal conversion unit 118 selectively. Moreover, as described in a step 206, the digitization conversion processing may be selectively performed on the data of the physiological information collected by the system management module 110 by the signal conversion unit 118, so as to convert the analogue data into a digital model for facilitating the data signal transmission of the message transmission model 112.

Then, as described in a step 208, a data comparison step is performed by the comparison unit 120 of the system management module 110 of the physiological information sensor 102, to determine that whether a digitalized datum of the physiological information is changed with respect to a previous digitalized datum of the same physiological information. When the datum of the data of the physiological information is the same as the previous datum, the system management module 110 will not transmit the datum to the message transmission module 112 and return to the step 204 for sensing the physiological information of the user, thereby preventing electric power from being consumed due to too many data transmissions

On the other hand, when the datum of the data of the physiological information is different from the previous datum, as described in a step 210, the datum of the physiological information can be transmitted to the message transmission module 112 by the system management module 110, and then can be transmitted to the data analysis device 104 by the message transmission module 112. In the step 210, the message transmission module 112 can transmit the datum of the physiological information to the data analysis device 104 through a wireless transmitter, e.g., a Zigbee wireless transmitter.

Then, as described in a step 212, the datum of the physiological information transmitted by the physiological information sensor 102 are analyzed and identified by the data analysis device 104, so as to determine whether the datum of the physiological information exceeds a predetermined alert value.

In the exemplary embodiment, which the application service system 106 of the physiology monitoring system 100 is a health care system, the data analysis device 104 can record and store the physiological information of the user and analyze various received physiological information groups of the user, such as body temperature, blood oxygen concentration, heartbeat, pressure, etc. Then, according to the physiological information groups, the data analysis device 104 can calculate data features, such as a maximum value, a minimum value, an average value, a root mean square value, a standard deviation, information entropy, a frequency, etc., corresponding to various physiological information groups. When any data feature of the physiological information group exceeds a corresponding predetermined alert value, the data analysis device 104 will determine that an alert condition event occurs.

When the alert condition event occurs, a step 214 is performed to use the data analysis device 104 to send out warning information and record the duration time of the alert condition event, i.e., the duration time of the warning information, for the subsequent analysis. The data analysis device 104 can send out the warning information by making sounds or sending a message to inform the user that an alert condition occurs. As described in a step 216, the data analysis device 104 then determines the critical condition event according to the alert condition event.

In the exemplary embodiment, to determining of the critical condition event may include the following two methods. The first method uses the duration time of the alert condition event as the basis of the determination threshold. In this method, the data analysis device 104 is used to determine whether the duration time of the alert condition event exceeds a predetermined duration time of the alert condition event. When the duration time of the alert condition event exceeds the predetermined duration time of the alert condition event, the data analysis device 104 determines that a critical condition event occurs. The data analysis device 104 may be used to transmit the critical condition event notice to the application service system 106. In one example, the predetermined duration time of the alert condition event may be 10 seconds, but is not limited thereto.

The second method determines the critical condition event by calculating with the use of a predetermined equation. In this exemplary embodiment, when the alert condition event occurs, the data analysis device 104 calculates the occurrence probability of the alert condition event corresponding to each critical event through the following equation (1), so as to obtain the possible occurrence probability value of each critical condition event under the occurrence of the alert condition event. Then, the data analysis device 104 compares the calculated possible occurrence probability values of the critical condition events and regards the critical condition event with the highest occurrence probability value as a determination result of the critical condition event.

For example, S may be defined as the alert condition event, in which S={S₁, S₂, S₃, . . . , Sₙ}. The space S includes data features of various physiological information. Each data feature of the physiological information, which exceeds the predetermined alert value, represents one alert condition event. For example, S₁ may represent the alert condition event that the standard deviation of the blood oxygen exceeds the predetermined alert value; and S₂ may represent the alert condition event that the maximum value of the heartbeat exceeds the predetermined alert value.

Additionally, E may be defined as the critical condition event, in which E={E₁, E₂, E₃, . . . , Eₙ}. The space E includes the combination of various critical condition events. For example, E₁ may represent the critical condition event of heart disease; E₂ may represent the critical condition event of coma; and E₃ may represent the critical condition event of falling accident, etc.

The determination method of the data analysis device 104 for the critical condition event is performed by calculating with the equation (1) to determine the critical condition event, which may occur when a certain alert condition event occurs. The alert condition events Sⱼ and Sₖ are taken as examples below. The data analysis device 104 calculates the occurrence probabilities of the alert condition events Sⱼ and Sₖ with respect to each critical condition event (Eᵢ) through the following equation (1) to obtain the occurrence probability of a certain critical condition event, which may occur. According to the calculation result, the critical condition event with the highest occurrence probability value is determined as the critical condition event, which occurs. The equation (1) is described as follows:

$$P ( E = {E}_{i} \vert {S}_{j} , {S}_{k} ) = \frac{ P ( {S}_{j} , {S}_{k} \vert {E}_{i} ) P ( {E}_{i} )}{{\sum }_{i = 1}^{n} P ( {S}_{j} , {S}_{k} \vert {E}_{i} ) P ( {E}_{i} )} \qquad ( 1 )$$

In the equation (1), P(Sⱼ,Sₖ|Eᵢ) are the occurrence probabilities of the alert condition events Sⱼ and Sₖ when the critical condition event Eᵢ occurs, and is a probability value, which is obtained in advance according to the experiment or the experience and is stored in a database. P(Eᵢ) is the occurrence probability of the critical condition event Eᵢ. Specifically, P(Eᵢ) is the number of previous occurrence times of the critical condition event Eᵢ being divided by the sum of the number of previous occurrence times of various critical condition events in the historical record. Σᵢ₌₁ⁿP(Sⱼ,Sₖ|E₁)P(E₁) represents the occurrence probability collection of various critical condition events E corresponding to the occurrence of the alert condition events Sⱼ and Sₖ. That is to say, the occurrence probability of each critical condition event E corresponding to Sⱼ and Sₖ is multiplied by the occurrence probability of each critical condition event E, and then the multiplied results are added up.

For example, if it is desired to obtain the critical condition event E, which may occur while the alert condition events Sⱼ and Sₖ occur simultaneously, the occurrence probability P(Eᵢ) of each critical condition event Eᵢ is discovered according to the historical record stored in advance. Then, the possible occurrence probability of each critical condition event Eᵢ corresponding to the alert condition events Sⱼ and Sₖ is counted; and the occurrence probability P(Sⱼ,Sₖ|Eᵢ) of the alert condition events Sⱼ and Sₖ in each critical condition event Eᵢ is counted. Therefore, the possible occurrence probability of each critical condition event Eᵢ can be obtained on the premise of the occurrence of the alert condition event. After the occurrence probability value of each critical condition event Eᵢ is obtained, the critical condition event with the highest probability value can be regarded as the inference result.

Then, the data analysis device 104 transmits the inference result to the application service system 106 to inform the user that the critical condition event may occur, or inform the persons or the medical staffs related to the user to process the critical condition event through a network or a message.

In one exemplary embodiment, the data analysis device 104 can define reference values of the data of each kind of physiological information respectively according to each data feature, and then regards the values, which are ±5% of the reference values, as critical indexes, i.e., predetermined alert values. However, in other exemplary embodiments, the predetermined alert value of each data feature can be adjusted according to the differences of the physiological information to be analyzed as well as the physiology monitoring targets. The critical indexes of the data features of the physiological information of the present invention are not limited to the values, which are ±5% of the reference values in the aforementioned exemplary embodiment.

In an exemplary example, the physiology monitoring method 200 is applied to the learning analysis of the user. As shown in FIG. 3, in the exemplary example, the physiological information sensor 102 is connected to the outer side surface of the mouse 126, and uses the information of the pressure applied to the mouse and the body temperature to carry out an analysis. This learning analysis method determines the physiology condition event of the user, which may occur, according to the received physiological information. The physiology condition event includes: a normal condition, a tired condition, an anxious condition and an off condition.

In the exemplary example, the data analysis device 104 can analyze various received physiological information groups of the user, such as body temperature, pressure applied to the mouse, blood oxygen concentration, etc., and calculate the data features, such as a duration time, an amount, a discrete value, an average value, etc., corresponding to various physiological information groups.

In one exemplary example, when any data feature of the physiological information group exceeds a predetermined alert value, the data analysis device 104 determines that an alert condition event occurs. When the alert condition event occurs, the step 214 is performed to use the data analysis device 104 to send out warning information and record the duration time of the alert condition event, i.e., the duration time of the warning information, for the subsequent analysis. The data analysis device 104 can send out the warning information by making sounds or sending a message to inform the user that an alert condition occurs. As described in the step 216, the data analysis device 104 then determines the physiology condition event according to the alert condition event.

In the exemplary example, when the alert condition event occurs, the data analysis device 104 calculates the occurrence probability of the alert condition event corresponding to each physiology condition event through the aforementioned equation (1), so as to obtain the possible occurrence probability value of each physiology condition event when this alert condition event occurs. Then, the data analysis device 104 compares the calculated possible occurrence probability values of the physiology condition events and regards the physiology condition event with the highest probability value as the determination result of the physiology condition event of the user.

For example, S may be defined as the alert condition event, in which S={S₁, S₂, S₃, . . . , Sₙ}. The space S includes data features of various kinds of physiological information, such as body temperature, blood oxygen concentration, pressure applied to the mouse, etc., in which the data features may be a duration time, an amount, a discrete value, an average value, etc. Each data feature of the physiological information represents one alert condition event. For example, S₁ may represent the alert condition event that the average value of the body temperature exceeds the predetermined alert value; and S₂ may represent the alert condition event that the amount of the pressure applied to the mouse exceeds the predetermined alert value.

Additionally, E may be defined as the physiology condition event, in which E={E₁, E₂, E₃, . . . , Eₙ}. The space E includes various physiology condition events. For example, E₁ may represent the physiology condition event that the user is tired; E₂ may represent the physiology condition event that the user is anxious; and E₃ may represent the physiology condition event that the user is off.

The determination method of the data analysis device 104 for the physiology condition event is performed by calculating with the equation (1) to determine the physiology condition event, which may occur when a certain alert condition event occurs. The alert condition events Sⱼ and Sₖ are taken as examples below. The data analysis device 104 calculates the occurrence probabilities of the alert condition events Sⱼ and Sₖ with respect to each critical condition event (Eᵢ) through the equation (1) to obtain the occurrence probability of a certain physiology condition event, which may occur. According to the calculation result, the critical condition event with the highest occurrence probability value is determined as the physiology condition event, which occurs.

In the exemplary example, P(Sⱼ,Sₖ|Eᵢ) in the equation (1) are the occurrence probabilities of the alert condition events Sⱼ and Sₖ when the physiology condition event Eᵢ occurs. P(Eᵢ) is the occurrence probability of the physiology condition event Eᵢ. Specifically, P(Eᵢ) is the number of previous occurrence times of the physiology condition event Eᵢ being divided by the sum of the number of previous occurrence times of various physiology condition events in the historical record. Σᵢ₌₁ⁿP(Sⱼ,Sₖ|Eᵢ)P(Eᵢ) represent the occurrence probability collection of various physiology condition events E corresponding to the occurrence of the alert condition events S and Sₖ. That is to say, the occurrence probability of each physiology condition event E corresponding to Sⱼ and Sₖ is multiplied by the occurrence probability of each physiology condition event E, and then the multiplied results are added up.

For example, if it is desired to obtain the physiology condition event E, which may occur while the alert condition events S and Sₖ occur simultaneously, the occurrence probability P(Eᵢ) of each physiology condition event Eᵢ is discovered according to the historical record stored in advance. Then, the possible occurrence probability of each physiology condition event Eᵢ corresponding to the alert condition events Sⱼ and Sₖ is counted; and the occurrence probability P(Sⱼ,Sₖ|Eᵢ) of the alert condition events Sⱼ and Sₖ in each physiology condition event Eᵢ is counted. Therefore, the possible occurrence probability value of each physiology condition event Eᵢ can be obtained on the premise of the occurrence of the alert condition event. After the occurrence probability value of each physiology condition event Eᵢ is obtained, the physiology condition event with the highest probability value can be regarded as the inference result.

Then, the data analysis device 104 transmits the inference result to the application service system 106 to inform the user that the physiology condition event may occur, or inform the persons related to the user to process the physiology condition event through a network or a message. For example, when it is determined that the user is tired, the persons related to the user, e.g., teachers or parents, can require the user to stand up to do some activities.

In the exemplary embodiment that the application service system 106 is a health care system, the application service system 106 can display the information of the critical condition event on the device used by the user to inform the user, or can inform the doctor or family of the user the message of this critical condition event by sending the message of this critical condition event through a network. Additionally, in the embodiment that the application service system 106 is a learning analysis system, the application service system 106 can display the information of the physiology condition event to inform the instructor, so as to facilitate the instructor to change the teaching content dynamically; or it can display a message or make sounds to inform the user to facilitate the user to return to the concentration learning condition.

Then, as described in a step 218, the application service system 106 provides the user with the corresponding service according to the physiology condition of the user provided by the data analysis device 104. In the step 218, according to the application field of the physiology monitoring method 200, the application service system 106 can provide different application services correspondingly. In the embodiment that the physiology monitoring method 200 is applied to the health care system, the data analysis device 104 can actively transmit a signal to inform the medical staffs or provide the user with the related health medical service through the application service system 106. On the other hand, in the embodiment that the physiology monitoring method 200 is applied to the learning analysis system, the data analysis device 104 can send a message to inform the application service system 106 to replace the teaching content or change the teaching method according to the determination result of the poor concentration of the user, so as to improve the teaching quality and increase the learning efficiency of the user.

Next, as described in a step 220, the data or the alert values changed in the previous steps are stored by the data analysis device 104, so as to update the historical record stored in the data analysis device 104. The changed data or the alert values can be used as reference data for the subsequent analysis and application. In one exemplary embodiment, in the historical record stored in the data analysis device 104, each kind of physiological information records 30 data and records data features of the 30 data at the same time, such as a frequency, a maximum value, a minimum value, an average value, a root mean square value, a standard deviation, information entropy and/or a duration time of an alert information, etc. After the step 220 is completed, the physiology monitoring method 200 proceeds to return to the step 204 of sensing the physiological information of the user to continue the sense and monitor of the physiology condition.

In one exemplary embodiment, the user may further make a feedback about whether the inference result of the data analysis device 104 is correct or not through a user interface of the application service system 106, so as to revise the historical database. If the inference result of the data analysis device 104 is correct, then the data can be added into the historical data. If the inference result of the data analysis device 104 is wrong, the user can abandon the data or retrieve the correct critical/physiology event, so that the application service system 106 can update the historical data according to the retrieved result.

On the other hand, in the step 216, when the data analysis device 104 determines no critical/physiology condition event occurs, the step 220 is performed to use the data analysis device 104 to store the data, so as to update the historical record stored in the data analysis device 104. Similarly, after the step 220 is completed, the physiology monitoring method 200 proceeds to return to the step 204 to continue the sense and monitor of the physiological information of the user.

Refer to FIG. 4 again. In the step 212, when the data of the physiological information does not exceed the predetermined alert value, the data analysis device 104 may display the data of the physiological information simply through its own display device. Alternatively, the data of the physiological information may be transmitted to the application service system 106 by the data analysis device 104 and displayed through the display device of the application service system 106 to inform the user.)

Then, as described in a step 224, after the data of the physiological information are displayed, the data analysis device 104 may be used to determine whether the data comply with the data in the historical record. When the data comply with the data in the historical record, the physiology monitoring method 200 proceeds to return to the step 204 of sensing the physiological information of the user. On the other hand, when the data do not comply with the data in the historical record, the step 220 is performed to use the data analysis device 104 to store the data, so as to update the historical record stored in the data analysis device 104. Similarly, after the step 220 is completed, the physiology monitoring method 200 proceeds to return to the step 204 to continue the sense and monitor of the physiological information of the user.

According to the aforementioned embodiments of the present invention, one advantage of the present invention is that the physiological information sensor of the present invention may be attached to an outer side surface of a device being used by a user through a connector. Accordingly, the physiological information sensor can be applied to various devices according to the demand of the user, thereby greatly increasing the application of the physiological information sensor and lowering the burden of the user.

According to the aforementioned embodiments of the present invention, another advantage of the present invention is that the physiology monitoring system and physiology monitoring method of the present invention can integrate a physiological information sensor and a data analysis device for physiological information with an application service system effectively. Accordingly, the sensed physiological information can be immediately analyzed and then the physiology condition of the user can be determined, so as to provide an application service needed by the user to achieve the effect of integrating the physiological information sensor with life.

As is understood by a person skilled in the art, the foregoing preferred embodiments of the present invention are illustrative of the present invention rather than limiting of the present invention. It is intended to cover various modifications and similar arrangements included within the spirit and scope of the appended claims, the scope of which should be accorded the broadest interpretation so as to encompass all such modifications and similar structure.

What is claimed is:

1. A physiology monitoring system, including:
a physiological information sensor, suitable for sensing at least one of physiological information of a user;
a data analysis device, suitable for receiving a plurality of data from the at least one physiological information from the physiological information sensor, calculating a plurality of data features of the data of the at least one physiological information, determining whether each of the data features has an alert condition event, calculating occurrence probabilities of a plurality of critical condition events or a plurality of physiology condition events by using the corresponding alert condition event(s), and determining a critical condition or a physiology condition of the user according to the occurrence probabilities of the critical condition events or the physiology condition events; and
an application service system, suitable for providing the user with a service according to the critical condition or the physiology condition of the user.

2. The physiology monitoring system according to claim 1, wherein the physiological information sensor includes:
a casing;
a physiological information sensing module, disposed within the casing and suitable for sensing the at least one physiological information;
a message transmission module, disposed within the casing and suitable for transmitting the received data to the data analysis device;
a system management module, disposed within the casing and suitable for collecting the data of the at least one physiological information and transmitting the data to the message transmission module; and
a connector, disposed on an outer side surface of the casing and suitable for connecting the casing to a device being used by the user.

3. The physiology monitoring system according to claim 1, wherein the application service system is a health care system, and is suitable for providing the user with a health care service according the critical condition of the user.

4. The physiology monitoring system according to claim 3, wherein the at least one physiological information includes body temperature, blood oxygen concentration, blood pressure and/or heartbeat, and the data features include a maximum value, a minimum value, an average value, a root mean square value, a standard deviation, information entropy and/or a frequency.

5. The physiology monitoring system according to claim 1, wherein the application service system is a learning analysis system and is suitable for facilitating learning efficiency of the user according to the physiology condition of the user.

6. The physiology monitoring system according to claim 5, wherein the at least one physiological information includes body temperature, blood oxygen concentration and/or pressure, and the data features include an average value, a duration time, an amount and/or a discrete value.

7. A physiology monitoring method, including:
using a physiological information sensor to sense at least one physiological information of a user;
using the physiological information sensor to compare a plurality of data of the at least one physiological information being sensed;
using a data analysis device to calculate a plurality of data features corresponding to the data of the at least one physiological information through the data of the at least one physiological information;
using the data analysis device to determine whether each of the data to features has an alert condition event;
using the data analysis device to calculate occurrence probabilities of corresponding critical condition events or a corresponding physiology condition events according to the alert condition event or the alert condition events when one alert condition event or a plurality of alert condition events occur among the data features;
using the data analysis device to determine a critical condition or a physiology condition of the user according to occurrence probability values of the critical condition events or the physiology condition events corresponding to the alert condition event or the alert condition events; and
using the data analysis device to transmit the critical condition or the physiology condition to an application service system.

8. The physiology monitoring method according to claim 7, wherein the physiological information sensor includes a casing, and a physiological information sensing module, a message transmission module and a system management module disposed within the casing, the system management module includes a timing unit, a signal conversion unit and a comparison unit, and the step of comparing the data includes:
using the timing unit to periodically require the physiological information sensing module to sense the at least one physiological information of the user;
using the signal conversion unit to convert the data from an analogue model to a digital model; and
using the comparison unit to compare the converted data being, so as to determine whether each of the data is changed with respect to a previous one of the data of the at least one physiological information.

9. The physiology monitoring method according to claim 7, wherein when each of the data features does not have the alert condition event, the data analysis device is used to display the data.

10. The physiology monitoring method according to claim 7, wherein when each of the data features does not have the alert condition event, the data analysis device is used to transmit the data to the application service system, and the data are displayed by the application service system.

11. The physiology monitoring method according to claim 7, wherein
when each of the data complies with the historical record, the physiology monitoring method returns to the step of sensing the at least one physiological information of the user; and
when each of the data does not comply with the historical record, the data analysis device uses each datum of these data to update the historical record is updated with each of the data by the data analysis device.

12. The physiology monitoring method according to claim 7, wherein the application service system is a health care system, the physiological information sensor is used to sense body temperature, blood oxygen concentration, blood pressure and/or heartbeat of the user, and the data analysis device is used to calculate a maximum value, a minimum value, an average value, a root mean square value, a standard deviation, information entropy and/or a frequency corresponding to the data of the at least one physiological information.

13. The physiology monitoring method according to claim 7, wherein the application service system is a learning analysis system, the physiological information sensor is used to sense body temperature, blood oxygen concentration and/or pressure of the user, and the data analysis device is used to calculate an average value, a duration time, an amount and/or a discrete value corresponding to the data of the at least one physiological information.

14. The physiology monitoring method according to claim 7, wherein the step of using the data analysis device to determine whether each of the data features has the alert condition event is performed by determine that whether each of the data features exceeds a corresponding predetermined alert value, and when one of the data features exceeds the corresponding predetermined alert value, the one of the data features has the alert condition event.

15. The physiology monitoring method according to claim 7, wherein the step of using the data analysis device to calculate occurrence probabilities of the corresponding critical condition events or physiology condition events according to the alert condition event or the alert condition events includes:
finding the occurrence probability of each of the critical condition events or the physiology condition events under the alert condition event or the alert condition events according to a historical record;
counting a possible occurrence probability of each of the critical condition events or the physiology condition events corresponding to the alert condition event or the alert condition events, and occurrence probabilities of the alert condition event or the alert condition events under each of the critical condition events or the physiology condition events; and
multiplying the occurrence probabilities of the critical condition events or the physiology condition events by the occurrence probabilities of the corresponding alert condition event or the corresponding alert condition events to obtain a calculating result, and then dividing the calculating result by the possible occurrence probability of each of the critical condition events or the physiology condition events corresponding to the alert condition event or the alert condition events.

16. The physiology monitoring method according to claim 7, wherein when the alert condition event or the alert condition events last more than a predetermined duration time, the data analysis device determines that the critical condition event or the physiology condition event occurs.
